(19) United States
(12) Patent Application Publication
(10) Pub. No.: US 2025/0136251 A1
(43) Pub. Date: May 1, 2025

(54) ADJUSTABLE OCEAN BUOY BRACKET FOR SUPPORTING SENSORS

(71) Applicant: Ningbo Institute of Oceanography, Ningbo (CN)

(72) Inventors: Fan LI, Ningbo (CN); Congying HE, Ningbo (CN); Shiwei WANG, Ningbo (CN); Kuanhong MENG, Ningbo (CN); Shuoqian MAO, Ningbo (CN); Qianwen SHAO, Ningbo (CN)

(21) Appl. No.: 19/008,404

(22) Filed: Jan. 2, 2025

Related U.S. Application Data
(63) Continuation of application No. PCT/CN2023/095489, filed on May 22, 2023.

(30) Foreign Application Priority Data
Jul. 1, 2022 (CN) 202210773465.5

(51) Int. Cl. B63B 22/00; B63B 59/00; B63B 79/10

(52) U.S. Cl. CPC B63B 22/00; B63B 59/00; B63B 79/10; B63B 2022/006

(57) ABSTRACT

An adjustable ocean buoy bracket for supporting sensors includes a support mechanism, a bearing mechanism, and an energy supply mechanism. The bearing mechanism is bolted to the upper surface of the support mechanism, and the energy supply mechanism is bolted inside the bearing mechanism. The buoy bracket further comprises a change mechanism. Through this change mechanism, the sensor performs a reciprocal motion for cleaning, thereby prolonging its service life.

### CROSS RERERENCE OF RELATED APPLICATIONS

This application is a continuation of International Application No. PCT/CN2023/095489, filed on May 22, 2023, entitled “Adjustable Oceanographic Buoy Support Capable of Carrying Sensors”, which claims priority of Chinese Patent Application No. 202210773465.5, filed on Jul. 1, 2022 in the China National Intellectual Property Administration (CNIPA), the entire contents of which is hereby incorporated by reference in its entireties.

### TECHNICAL FIELD

The present invention relates to the field of ocean buoy technology and, more particularly, to an adjustable ocean buoy bracket for supporting sensors.

### BACKGROUND OF THE INVENTION

Ocean buoys are modern observation facilities that reliably collect ocean environmental data around the clock and enable automatic data acquisition, labeling, and transmission. Together with satellites, aircraft, research vessels, submersibles, and sonar detection equipment, ocean buoys form a core component of the modern ocean environment monitoring system. Multiple sensors are mounted on the buoy bracket, which remains in harsh conditions for extended periods, reducing sensor lifespan, making them prone to damage, and hindering their cleanliness.

It should be noted that the information disclosed in this Background section is provided only to enhance the understanding of the invention and should not be construed as indicating that this information is part of the prior art known to those skilled in the art.

### SUMMARY OF THE INVENTION

In order to address problems in the prior art, the present invention provides an adjustable ocean buoy bracket for supporting sensors. By including three suction filter heads, debris-free seawater can be drawn for detection. A drying plate prevents accumulated water on the upper portion from affecting measurements, thereby reducing errors. Under the action of a photovoltaic panel and a motor-driven roller, the bracket can be adjusted to achieve an optimal working angle. With a cam block driven by a motor, an external force can be applied to a moving rod, causing the sensor measurement end to move back and forth. Finally, under the force of a return spring, the sensor measurement end rapidly returns to its initial position and is cleaned and dried in a cleaning chamber.

To achieve the above purpose, the present invention adopts the following technical solution:

An adjustable ocean buoy bracket for supporting sensors, including a support mechanism, a bearing mechanism, and an energy supply mechanism. The bearing mechanism is bolted to the upper surface of the support mechanism. The energy supply mechanism is bolted inside the bearing mechanism. The buoy bracket further includes a change mechanism to maintain a favorable operating state for the sensors. The change mechanism includes a loading box, a covering plate, a storage ring, a connecting tube, a cleaning chamber, a motor, a bearing shaft, a cam block, a moving rod, a sensor measurement end, a concave-convex plate, and a return spring. The loading box is bolted to the center of the upper surface of the support mechanism, and the covering plate is detachably connected to the top surface of the loading box. A storage ring is bolted to the center of the top surface of the covering plate, with one side of the storage ring communicating with the cleaning chamber through the connecting tube. The motor is disposed inside the storage ring, and the output shaft of the motor is bolted to the bearing shaft. The cam block is keyed to the bearing shaft, and the concave-convex plate is arranged on both sides of the bearing shaft, connected to the loading box by bolts. One side of the cam block is in rolling contact with the moving rod. One end of the moving rod is equipped with the sensor measurement end. The moving rod passes through the concave-convex plate, and the return spring is installed at the through-hole between the moving rod and the concave-convex plate.

On the basis of the foregoing technical solution, the support mechanism includes a buoy body, a control compartment, an anchoring frame, and a suction filter head. The buoy body is installed on the upper surface of the control compartment. The anchoring frame is bolted to the lower portion of the buoy body. The suction filter head is detachably connected to the lower surface of the control compartment.

On the basis of the foregoing technical solution, the bearing mechanism includes support legs, a bearing disc, a blocking frame, a humidity detection head, a drying plate, and a rotating slot. The support legs are bolted at the four corners of the upper surface of the control compartment, and the upper ends of the support legs are bolted to the bearing disc. The blocking frame is installed along the edge of the bearing disc, and the drying plate is bonded at the center of the upper surface of the bearing disc. The humidity detection head is installed at one corner of the drying plate, and the rotating slot is formed where the support legs contact the energy supply mechanism.

On the basis of the foregoing technical solution, the energy supply mechanism includes a photovoltaic panel and a rotating shaft. The rotating shaft is rotatably connected between the support legs, and the photovoltaic panel is installed on the upper portion of the rotating shaft.

On the basis of the foregoing technical solution, a shock-absorbing foam is bonded to the internal surface of the control compartment, and three suction filter heads are provided in total.

On the basis of the foregoing technical solution, each support leg is hollow and has a wall thickness of 2-5 cm, and the drying plate is reusable.

On the basis of the foregoing technical solution, an electric roller is installed at the contact position between the photovoltaic panel and the rotating slot, and aluminum alloy frames are arranged along the four sides of the photovoltaic panel.

On the basis of the foregoing technical solution, a smooth outer sleeve is installed at the contact position between the loading box and the moving rod, and the cleaning chamber is a combined device comprising a pressure pump in the front section and a blower in the rear section.

On the basis of the foregoing technical solution, a seal is formed between the concave-convex plate and the loading box, and the moving rod is made of stainless steel.

On the basis of the foregoing technical solution, five cam blocks are arranged in total, and five pairs of moving rods are provided in total.

Compared with the prior art, the advantages of the present invention are as follows: Three suction filter heads absorb debris-free seawater for detection; the drying plate prevents accumulated water from affecting measurements and causing larger errors; the photovoltaic panel and electric roller enable angle adjustment for optimal operation; the cam blocks driven by the motor apply external force to the moving rods, achieving back-and-forth movement of the sensor measurement ends; and finally, with the return spring, the sensor measurement ends quickly revert to the initial state and are cleaned and dried in the cleaning chamber. Additional technical features and advantages of the present invention will be further clarified in the following description or will become apparent through implementation of the invention.

### BRIEF DESCRIPTION OF DRAWINGS

The accompanying drawings provide a further understanding of the present invention, form part of the specification, and, together with the following detailed description, serve to explain the invention without limiting its scope. In the drawings:

FIG. 1 is a perspective view of the adjustable ocean buoy bracket for supporting sensors according to the present invention;

FIG. 2 is a front view of the adjustable ocean buoy bracket for supporting sensors according to the present invention;

FIG. 3 is a perspective view of the change mechanism of the adjustable ocean buoy bracket for supporting sensors according to the present invention;

FIG. 4 is a schematic internal view of the change mechanism of the adjustable ocean buoy bracket for supporting sensors according to the present invention;

FIG. 5 is a perspective view of a support leg of the adjustable ocean buoy bracket for supporting sensors according to the present invention.

### CHARACTER REFERENCES

1, support mechanism; 101, buoy body; 102, control compartment; 103, anchoring frame; 104, suction filter head; 2, bearing mechanism; 201, support leg; 202, bearing disc; 203, blocking frame; 204, humidity detection head; 205, drying plate; 206, rotating slot; 3, energy supply mechanism; 301, photovoltaic panel; 302, rotating shaft; 4, change mechanism; 401, loading box; 402, covering plate; 403, storage ring; 404, connecting tube; 405, cleaning chamber; 406, motor; 407, bearing shaft; 408, cam block; 409, moving rod; 410, sensor measurement end; 411, concave-convex plate; 412, return spring.

### DETAILED DESCRIPTION OF THE INVENTION

The drawings provided in the embodiments of the present invention will be referenced for a clear and complete description of the technical solutions therein. It should be understood that the embodiments described represent only some examples rather than all possible embodiments of the present invention.

In the description of the present invention. the terms “upper,” “lower,” “front,” “back,” “left,” “right,” “top,” “bottom,” “inner,” “outer,” and the like refer to directions or positions based on those shown in the figures. Such expressions are meant to facilitate the description and streamline the disclosure and do not indicate or imply that any device or element must be constructed or operated in a specified orientation. They should therefore not be construed as limiting the invention in any manner.

### EMBODIMENTS

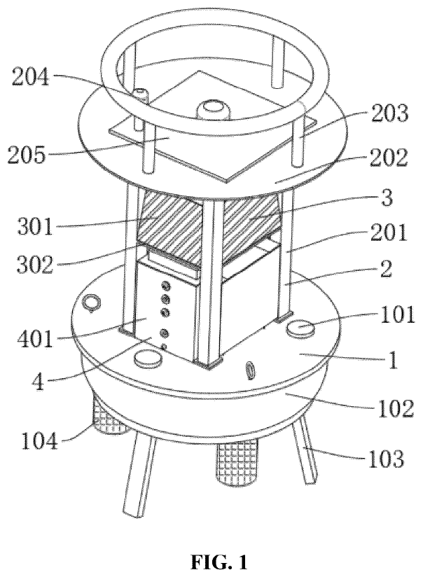

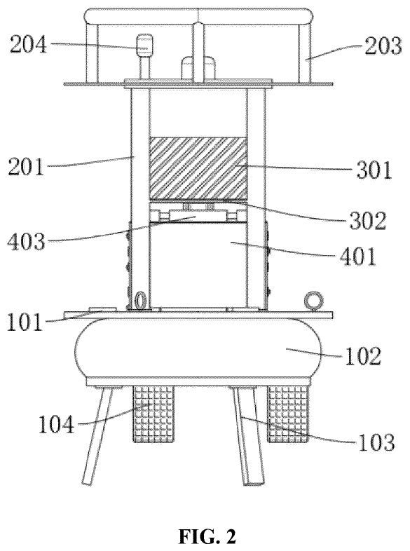

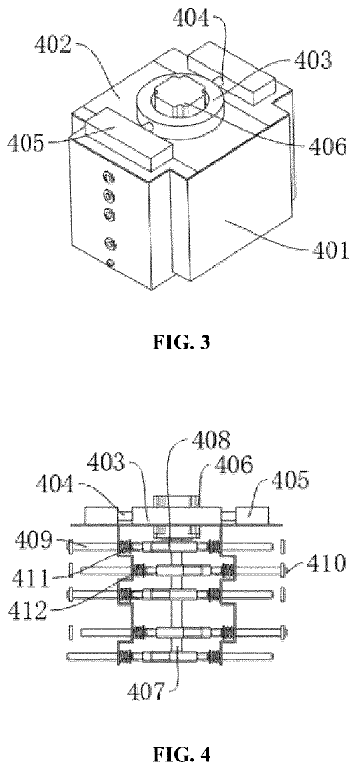

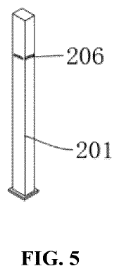

Referring to FIGS. 1-5, the present invention provides an adjustable ocean buoy bracket for supporting sensors. The bracket includes a support mechanism 1, a bearing mechanism 2, and an energy supply mechanism 3. The bearing mechanism 2 is bolted to the upper surface of the support mechanism 1. and the energy supply mechanism 3 is bolted inside the bearing mechanism 2. The bracket further includes a change mechanism 4 that maintains optimal sensor conditions. The change mechanism 4 includes a loading box 401, a covering plate 402, a storage ring 403, a connecting tube 404, a cleaning chamber 405, a motor 406, a bearing shaft 407, a cam block 408, a moving rod 409, a sensor measurement end 410, a concave-convex plate 411, and a return spring 412. The loading box 401 is bolted to the center of the upper surface of the control compartment 102. The covering plate 402 is detachably connected to the top surface of the loading box 401 for maintaining and diagnosing internal components. The storage ring 403 is bolted at the center of the covering plate 402, with one side of the storage ring 403 communicating with the cleaning chamber 405 through the connecting tube 404. The motor 406 is disposed inside the storage ring 403 and can drive the cam block 408 in rotation. The output end of the motor 406 is bolted to the bearing shaft 407, and the cam block 408 is keyed onto the bearing shaft 407 to apply force against the roller at one end of the moving rod 409. The concave-convex plate 411 is arranged on both sides of the bearing shaft 407 and are bolted to the loading box 401. One side of the cam block 408 is in rolling contact with the moving rod 409, and one end of the moving rod 409 is provided with the sensor measurement end 410. The moving rod 409 extends through the concave-convex plate 411, with the return spring 412 installed inside the through-hole between the moving rod 409 and the concave-convex plate 411, to rapidly restore the moving rod 409 to the initial position of the moving rod 309.

In the above embodiments, the support mechanism 1 includes a buoy body 101, a control compartment 102, an anchoring frame 103, and a suction filter head 104. The buoy body 101 is installed on the upper surface of the control compartment 102, and the anchoring frame 103 is bolted to the lower portion of the buoy body 101 to keep it stable. The suction filter head 104 is detachably connected to the lower surface of the control compartment 102 to prevent debris from entering and to draw seawater for testing. The bearing mechanism 2 includes support legs 201, a bearing disc 202, a blocking frame 203, a humidity detection head 204, a drying plate 205, and a rotating slot 206. The support legs 201 are bolted at the four corners of the upper surface of the control compartment 102 to maintain stability for upper components. The upper ends of the support legs 201 are bolted to the bearing disc 202, whose edge is fitted with the blocking frame 203. The drying plate 205 is bonded to the center of the bearing disc 202. and one corner of the drying plate 205 is equipped with the humidity detection head 204 to measure air humidity. The rotating slot 206, formed where the support legs 201 contact the photovoltaic panel 301. provides a track for rotation. The energy supply mechanism 3 includes the photovoltaic panel 301 and a rotating shaft 302. The rotating shaft 302 is rotatably connected between the support legs 201, and the photovoltaic panel 301 is installed on the upper portion of the rotating shaft 302. Shock-absorbing foam is bonded to the internal surface of the control compartment 102 to protect internal components from seawater impact. Three suction filter heads 104 are provided to simultaneously take in seawater. Each support leg 201 is hollow with a wall thickness of 2-5 cm to accommodate the rotating slot 206, and the drying plate 205 is reusable, enhancing the accuracy of the humidity detection head 204. An electric roller is installed at the contact position between the photovoltaic panel 301 and the rotating slot 206 for angle adjustment, and aluminum alloy frames are arranged along the four edges of the photovoltaic panel 301 to prevent corrosion-induced detachment. A smooth outer sleeve is installed where the loading box 401 contacts the moving rod 409 to assist the rod's reciprocating motion. The cleaning chamber 405 is a combined device comprising a pressure pump in its front section and a blower in its rear section, which delivers cleaning solution from the storage ring 403 to the surface of the sensor measurement end 410 and then blows it dry. A seal is formed between the concave-convex plate 411 and the loading box 401 to prevent seawater intrusion; the moving rod 409 is made of stainless steel. Five cam blocks 408 are provided in total, driving five pairs of moving rods 409 so that the sensor measurement ends 410 can be switched continuously.

During operation, the suction filter head 104 absorbs seawater for water-quality testing, while the humidity detection head 204 measures air humidity. The photovoltaic panel 301 can change angles to achieve an optimal working position. Under the action of the cam block 408, the sensor measurement ends 410 repeatedly shift positions to be cleaned in the cleaning chamber 405, thereby ensuring that the sensor measurement ends 410 remain in their optimal operating state.

Although certain embodiments have been described in detail by way of example, those skilled in the art will appreciate that these examples are merely illustrative and not restrictive of the present invention's scope. Various modifications can be made without departing from the scope and spirit of the present invention, which is defined by the appended claims.

1. An adjustable ocean buoy bracket for supporting sensors, comprising a support mechanism (1), a bearing mechanism (2), and an energy supply mechanism (3), wherein the bearing mechanism (2) is bolted to an upper surface of the support mechanism (1), and the energy supply mechanism (3) is bolted inside the bearing mechanism (2), characterized in that the adjustable ocean buoy bracket further comprises a change mechanism (4) for maintaining favorable operating conditions for the sensors, the change mechanism (4) comprising a loading box (401), a covering plate (402), a storage ring (403), a connecting tube (404), a cleaning chamber (405), a motor (406), a bearing shaft (407), a cam block (408), a moving rod (409), a sensor measurement end (410), a concave-convex plate (411), and a return spring (412); wherein the loading box (401) is bolted to a central position of an upper surface of the support mechanism (1); the covering plate (402) is detachably connected to an upper surface of the loading box (401); the storage ring (403) is bolted to a central position of the covering plate (402), one side of the storage ring (403) communicates with the cleaning chamber (405) through the connecting tube (404); the motor (406) is disposed inside the storage ring (403); an output end of the motor (406) is bolted to the bearing shaft (407); the cam block (408) is keyed onto the bearing shaft (407); the concave-convex plate (411) is arranged on both sides of the bearing shaft (407) and bolted to the loading box (401); one side of the cam block (408) is in rolling contact with the moving rod (409), one end of the moving rod (409) is provided with the sensor measurement end (410), the moving rod (409) passes through the concave-convex plate (411), and the return spring (412) is installed on an interior side of the through-hole between the moving rod (409) and the concave-convex plate (411).

2. The adjustable ocean buoy bracket for supporting sensors according to claim 1, characterized in that the support mechanism (1) comprises a buoy body (101), a control compartment (102), an anchoring frame (103), and a suction filter head (104); wherein the buoy body (101) is installed on an upper surface of the control compartment (102); the anchoring frame (103) is bolted to a lower portion of the buoy body (101); and the suction filter head (104) is detachably connected to a lower surface of the control compartment (102).

3. The adjustable ocean buoy bracket for supporting sensors according to claim 2, characterized in that the bearing mechanism (2) comprises support legs (201), a bearing disc (202), a blocking frame (203), a humidity detection head (204), a drying plate (205), and a rotating slot (206); wherein the support legs (201) are bolted at four corners of an upper surface of the control compartment (102); the bearing disc (202) is bolted to upper ends of the support legs (201); the blocking frame (203) is installed along an edge of the bearing disc (202); the drying plate (205) is bonded at a central position of an upper surface of the bearing disc (202); one corner of the drying plate (205) is provided with the humidity detection head (204); and the rotating slot (206) is formed at a contact position between the support legs (201) and the energy supply mechanism (3).

4. The adjustable ocean buoy bracket for supporting sensors according to claim 3, characterized in that the energy supply mechanism (3) comprises a photovoltaic panel (301) and a rotating shaft (302); the rotating shaft (302) is rotatably connected between the support legs (201); and the photovoltaic panel (301) is installed on an upper portion of the rotating shaft (302).

5. The adjustable ocean buoy bracket for supporting sensors according to claim 2, characterized in that shock-absorbing foam is bonded to an internal surface of the control compartment (102), and three suction filter heads (104) are provided.

6. The adjustable ocean buoy bracket for supporting sensors according to claim 3, characterized in that interiors of the support legs (201) are hollow with a wall thickness of 2-5 cm; and the drying plate (205) is reusable.

7. The adjustable ocean buoy bracket for supporting sensors according to claim 4, characterized in that an electric roller is installed at a contact position between the photovoltaic panel (301) and the rotating slot (206); and aluminum alloy frames are arranged along four edges of the photovoltaic panel (301).

8. The adjustable ocean buoy bracket for supporting sensors according to claim 1, characterized in that a smooth outer sleeve is installed at a contact position between the loading box (401) and the moving rod (409); and the cleaning chamber (405) is a combined device comprising a pressure pump in a front portion and a blower in a rear portion.

9. The adjustable ocean buoy bracket for supporting sensors according to claim 1, characterized in that a seal is formed between the concave-convex plate (411) and the loading box (401); and the moving rod (409) is made of stainless steel.

10. The adjustable ocean buoy bracket for supporting sensors according to claim 1, characterized in that five cam blocks (408) are provided in total; and five pairs of moving rods (409) are provided in total.
